(19) United States
(12) Patent Application Publication
(10) Pub. No.: US 2017/0311823 A1
(43) Pub. Date: Nov. 2, 2017

(54) OPTICAL TRIGGER FOR MEASUREMENT

(71) Applicant: Nonin Medical, Inc., Plymouth, MN (US)

(72) Inventors: Gregory J. RAUSCH, Minnetonka, MN (US); Dirk Robert HELGEMO, Shakopee, MN (US)

(21) Appl. No.: 15/529,743

(22) PCT Filed: Nov. 24, 2015
(86) PCT No.: PCT/US2015/062520

§ 371 Date: May 25, 2017

Related U.S. Application Data
(60) Provisional application No. 62/084,408, filed on Nov. 25, 2014.

(51) Int. Cl. A61B 5/024; A61B 5/08; A61B 5/1455; A61B 5/00

(52) U.S. Cl. CPC A61B 5/02416; A61B 5/7292; A61B 5/0816; A61B 5/14551

(57) ABSTRACT

A method includes emitting optical energy, detecting a feature, emitting optical energy, and determining a physiological parameter. The method includes emitting optical energy having spectral content in a first range. The first range includes green light or infrared light. The optical energy is directed at tissue using a wearable module. The method includes detecting a feature in a photoplethysmogram signal. The feature has spectral content corresponding to optical energy in the first range. In response to detecting the feature, the method includes emitting optical energy having spectral content in a second range. The second range is different from the first range. The method includes determining a physiological parameter using the optical energy in the second range.

### CLAIM OF PRIORITY

This patent application claims the benefit of priority of U.S. Provisional Patent Application Ser. No. 62/084,408, entitled OPTICAL TRIGGER FOR MEASUREMENT, filed on Nov. 25, 2014, which is hereby incorporated by reference herein in its entirety.

### BACKGROUND

A variety of physiological parameters can be used to provide a measure of health for a patient. One such measurement is oxygen saturation (commonly referred to as SpO2) which relates to a measure of oxygenation of blood. Another example includes regional oxygen saturation (commonly referred to as rSO2) which relates to oxygenation of a region or tissue. Oxygenation can be determined using a system of optical emitters and optical detectors along with suitable processing.

Accurate measurement of oxygenation can be very important for health or safety. Accuracy of the oxygenation measurement is influenced by various factors, including the timing of measurement. Approaches to determining or selecting timing have been inadequate.

### OVERVIEW

The present inventors have recognized, among other things, that a problem to be solved can include determining synchronization for an oxygenation measurement. The present subject matter can help provide a solution to this problem, such as by identifying an optically detected signal that is suitable for triggering. In addition, an example of the present subject matter can be configured to determine a respiration (or ventilation) rate.

A method includes emitting optical energy, detecting a feature, emitting optical energy, and determining a physiological parameter. The method includes emitting optical energy having spectral content in a first range. The first range includes green light or infrared light. The optical energy is directed at tissue using a wearable module. The method includes detecting a feature in a photoplethysmogram signal. The feature has spectral content corresponding to optical energy in the first range. In response to detecting the feature, the method includes emitting optical energy having spectral content in a second range. The second range is different from the first range. The method includes determining a physiological parameter using the optical energy in the second range.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a flow chart of a method, according to one example.

FIG. 2 illustrates a flow chart of a method, according to one example.

FIG. 3 illustrates a waveform corresponding to a method, according to one example.

FIG. 4A illustrates a block diagram of a system, according to one example.

FIG. 4B illustrates a block diagram of a system, according to one example.

### DETAILED DESCRIPTION

An example of the present subject matter entails using light of a particular wavelength for identifying a physiological event for use with a measurement of a parameter based on a photoplethysmogram signal.

Green light or infrared light can be used to detect a feature (in morphology) and the timing of that feature can be used as a trigger for an optical modulation sequence. The optical modulation sequence provides data for determining oxygenation according to an algorithm.

In one example, green light (or infrared light) can be used to detect a feature and then trigger acquisition of a physiological measurement. Acquisition of the physiological measurement can include evaluating an equation or conducting processing according to an algorithm. The physiological measurement can include emitting light (of one or more selected wavelengths) and in response to the light, generating a signal using a detector and processing the signal according to an algorithm.

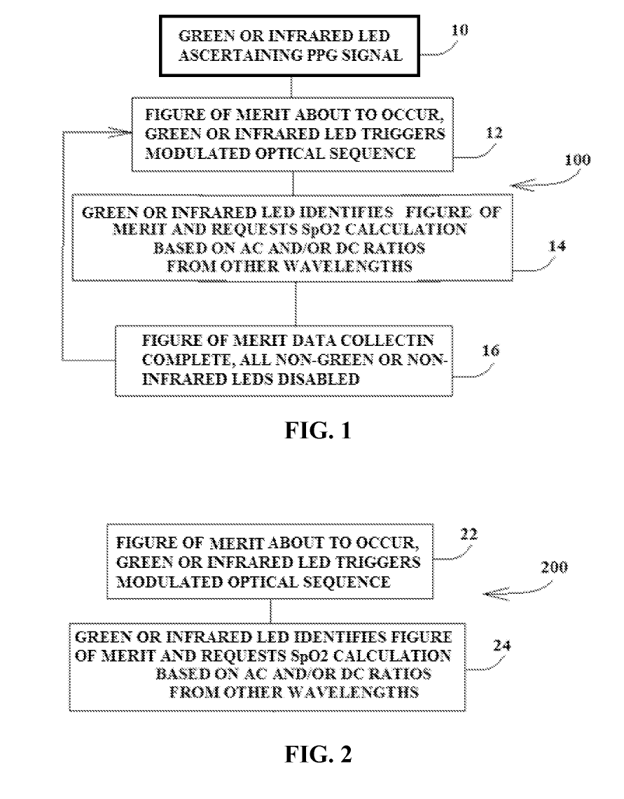

FIG. 1 includes a flow chart of method 100 according to one example. Method 100, at 10, includes using green light (or infrared light) to ascertain a photoplethysmogram signal. In one example, this includes emitting green or infrared light at tissue and in other examples, this includes emitting light of a color other than green or other than infrared. The light is emitted using a body-worn device affixed to the user. The light can be detected by reflection or by transmission through the tissue.

At 12, method 100 includes monitoring for occurrence of a figure of merit. This can include monitoring for a signal feature using a photodetector having sensitivity to green light or infrared light. Upon detecting occurrence, method 100 includes triggering a sequence of modulated pulses. The modulated pulses, which can be at other frequencies and can have an independent timing sequence, are emitted at the tissue and detected using the photodetector or other mechanism. The measure of oxygenation is determined based, in part, on the signals received from the detector.

At 14, method 100 includes using the green light (or infrared light) to identify a figure of merit and request calculation of oxygenation based on measured data. The measured data can include any combination of AC values, DC values, ratios, or other signal parameters.

At 16, method 100 includes completion of data collection and extinguishing power to the non-green emitters (or non-infrared emitters). The non-green emitters (or non-infrared emitters) are those emitters used for determining the measure of oxygenation.

FIG. 2 includes a flow chart of method 200 according to one example. Method 200, at 22, includes monitoring for occurrence of a figure of merit. This can include monitoring for a signal feature using a photodetector having sensitivity to green light or infrared light.

At 24, method 200 includes using the green light (or infrared light) to identify a figure of merit and triggering calculation of oxygenation based on the measured data. The measured data can include any combination of AC values, DC values, ratios, or other signal parameters.

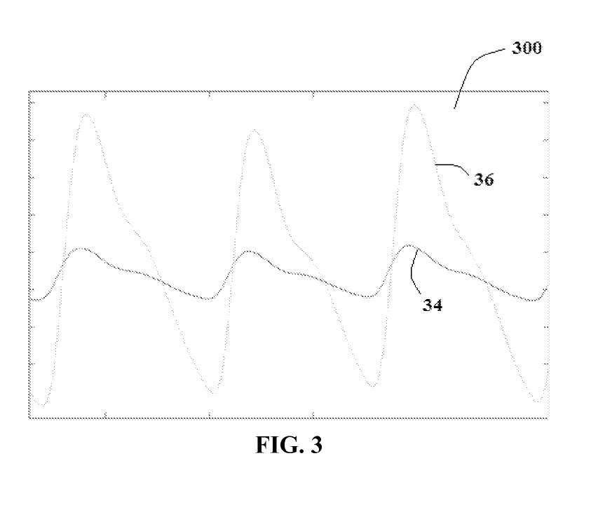

FIG. 3 illustrates absorption spectra 300 according to one example. Spectra 300 illustrates amplitude as a function of time. Data 36 illustrates amplitude of green light (or infrared light) and data 34 illustrates amplitude of non-green light (or non-infrared light), as measured by suitable detectors. In one example, the non-green light (or non-infrared light) can be viewed as IR wavelength.

As shown in the figure, data 36 and data 34 are substantially in phase and notably, the green light (or infrared light) amplitude is substantially greater than that of the non-green (or non-infrared) amplitude.

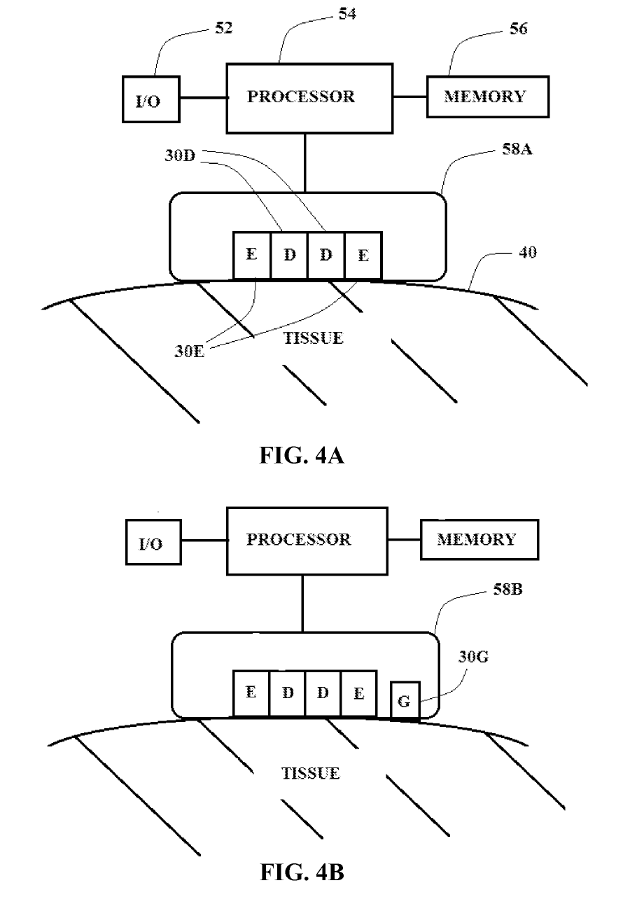

FIGS. 4A and 4B illustrate devices, according to various examples. FIG. 4A includes a block diagram of a system configured for affixation to tissue 40 at a site. The system is non-invasive and includes input/output module 52, processor 54, memory 56, and optical module 58A (having emitters 30E and optical detectors 30D).

Input/output module 52 can include a power switch, a mode control switch, a display, a user-control, a touch-screen, an indicator light, or other interface elements that enable a user to interact with system 50. Input/output module 52 can include a wireless interface to allow communication with a remote device.

Processor 54 can include an analog processor. In one example, processor 54 includes a digital processor and is configured to execute instructions for implementing an algorithm. The instructions and data can be stored in memory 56. Processor 54 can include an analog front end having an amplifier, a filter, a sample and hold circuit, an analog-to-digital converter (ADC), a digital-to-analog converter (DAC), an LED driver, or other modules.

Emitter 30E can include a light emitting diode (LED) configured to emit light of a selected wavelength and power. In the example illustrated, one or more of emitters 30E can be modulated to emit light having a green color or light in the infrared spectrum. Detector 30D can include a photodiode.

Light energy emitted by optical emitter 30E can be directed to reflect or pass through tissue. Light detected by optical detector 30D can be suitably processed to generate selected data in accordance with various examples of the present subject matter.

The system can be configured for wearing on a body. In this example, the system is powered by a portable power supply, such as a battery. The system can be affixed to a garment, a patch, or clamp device that remains in close proximity to the body for an extended duration.

FIG. 4B includes a block diagram of a system configured for affixation to tissue 40 at a site. The system is non-invasive and includes certain elements in common with that shown in FIG. 4A and further includes optical module 58B. Optical module 58B includes two emitters 30E and two optical detectors 30D and includes emitter 30G. Emitter 30G is configured to emit light (having a green color or in the infrared spectrum) at tissue 40.

Various figures of merit can be associated with the green light (or non-infrared light). A figure of merit can be viewed as a particular morphological feature that can be identified in the photoplethysmogram signal. For example, a figure of merit can include a peak value, an average value, a threshold crossing, an inflection point, an area under a curve, or a derivative value.

Upon detecting an occurrence of the figure of merit, one example of the present subject matter includes triggering excitation of other emitters, detecting light using a photodetector, and processing a signal based on the detected light to derive a physiological measurement (such as oxygenation or such as respiration rate).

In another example, upon detecting an occurrence of the figure of merit, the present subject matter includes triggering calculation of a physiological measurement (such as oxygenation) based on non-green (or non-infrared) light detected using a photodetector. The non-green (or non-infrared) light can include light having more than one wavelength.

A non-green light used for noninvasive measurements has a wavelength in the range of 600 to 1500 nm. At these wavelengths, absorption of light in tissue is low. Infrared light can have a wavelength of 850-950 nm. According to other examples, infrared light can have a wavelength of 700 nm-1000 nm. In general, infrared light can penetrate tissue to a depth greater than that of green light.

Low levels of absorption are critical for transmittance-based measurements and are not as critical for reflectance-based measurements of oxygenation.

Light having wavelengths in the range of 500 to 600 nm is of little value for discrimination of chromophores but can provide a strong signal for ascertaining a photoplethysmogram signal in high signal-to-noise environments.

An example of a high signal-to-noise environments entails measurement of oxygenation at a tissue site away from the finger where perfusion may be compromised.

A green light (or infrared light) emitting LED can be used to identify the figure of merit in the photoplethysmogram signal, and thus aid in identifying the relevant portion of the photoplethysmogram signal for use of the other wavelength data in the calculation of oxygenation.

One example of the present subject matter enables lower power consumption (therefore, smaller battery and commensurate decrease in form factor) with good signal-to-noise performance, thereby enabling SpO2 measurements from locations that are otherwise unusable.

One example includes a method. The method can include emitting optical energy having spectral content in a first range. The first range includes green light or includes infrared light. The optical energy is directed at tissue using a wearable module. The method includes detecting a feature in a photoplethysmogram signal. The feature can have spectral content corresponding to optical energy in the first range. In response to detecting the feature, the method includes emitting optical energy having spectral content in a second range. The second range is different from the first range. The method includes determining a physiological parameter using the optical energy in the second range. In one example, measured data corresponding to a feature of interest (such as determined by green light, infrared light, or light of other colors) is processed using an algorithm. The algorithm can be configured to evaluate an equation.

The method can also include performing a calculation using an algorithm to determine the physiological parameter. One example includes triggering a modulation sequence of an optical element. The first range can include light having a wavelength between 500 nm and 600 nm. The first range light can have a period of approximately 13.3 milliseconds. The second range light can include a wavelength between 600 nm and 1500 nm. The second range light can include an infrared wavelength. The second range pulsed light can include light having wavelengths corresponding to 660 nm, 730 nm, 810 nm, and 910 nm.

The pulse can have a duration of approximately 200 microseconds. The photoplethysmogram signal can be used for detecting a pulse component associated with a heart. In addition, the method can be used for determining blood oxygenation.

### Various Notes & Examples

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A method comprising:
emitting optical energy having spectral content in a first range, the first range including green or infrared light, the optical energy directed at tissue using a wearable module;
detecting a feature in a photoplethysmogram signal, the feature having spectral content corresponding to optical energy in the first range;
in response to detecting the feature, emitting optical energy having spectral content in a second range, the second range different from the first range; and
determining a physiological parameter using the optical energy in the first range, the second range, or in both the first and second range.

2. The method of claim 1 wherein determining the physiological parameter includes performing a calculation using an algorithm to determine the parameter.

3. The method of claim 1 wherein determining the physiological parameter includes triggering a modulation sequence of an optical element.

4. The method of claim 1 wherein emitting optical energy having spectral content in the first range includes emitting light having a wavelength between 500 nm and 600 nm.

5. The method of claim 1 wherein emitting optical energy having spectral content in the first range includes emitting light at a period of approximately 13 milliseconds.

6. The method of claim 1 wherein emitting optical energy having spectral content in the second range includes emitting light having wavelengths between 600 nm and 1500 nm.

7. The method of claim 1 wherein emitting optical energy having spectral content in the second range includes emitting light having an infrared wavelength.

8. The method of claim 1 wherein emitting optical energy having spectral content in the second range includes emitting pulsed light having wavelengths corresponding to 660 nm, 730 nm, 810 nm, and 910 nm.

9. The method of claim 1 wherein emitting optical energy having spectral content in the second range includes emitting pulsed light, wherein a pulse has a duration of approximately 100-500 microseconds.

10. The method of claim 1 wherein detecting the feature in the photoplethysmogram signal includes detecting a pulse component associated with a heart.

11. The method of claim 1 determining the physiological parameter includes determining blood oxygenation.

12. The method of claim 1 determining the physiological parameter includes determining respiration rate.

13. A method comprising:
emitting optical energy having spectral content in a first range, the first range including green light or infrared light, the optical energy directed at tissue using a wearable module;
detecting a feature in a photoplethysmogram signal, the feature having spectral content corresponding to optical energy in the first range;
in response to detecting the feature, detecting optical energy having spectral content in a second range; and
determining a physiological parameter using the optical energy in the first range, the second range, or in both the first and second range.

14. The method of claim 13 wherein the second range is different from the first range.

15. The method of claim 13 further including, in response to detecting the feature, detecting optical energy having spectral content in a third range; and
determining the physiological parameter using the optical energy in at least one of the first range, the second range, and the third range.

16. The method of claim 13 wherein determining the physiological parameter includes triggering a modulation sequence of at least one optical element.
